(19) United States
(12) Patent Application Publication
(10) Pub. No.: US 2018/0311486 A1
(43) Pub. Date: Nov. 1, 2018

(54) APPARATUS AND METHOD FOR MANUFACTURING MICRONEEDLE PATCH, AND MICRONEEDLE PATCH MANUFACTURED BY SAME

(71) Applicant: C&TECH CORPORATION, Hwaseong-si (KR)

(72) Inventor: Han Wook PARK, Seoul (KR)

(21) Appl. No.: 15/769,107

(22) PCT Filed: Aug. 12, 2016
(86) PCT No.: PCT/KR2016/008923

§ 371 Date: Apr. 18, 2018

(30) Foreign Application Priority Data
Dec. 4, 2015 (KR) 10-2015-0172153

(51) Int. Cl. A61M 37/00; A61K 9/00; A61K 9/70; A61K 47/36; A61K 8/02; A61K 8/73; B29C 41/32; B29C 41/00; B29C 41/26; B29C 41/30

(52) U.S. Cl. CPC A61M 37/0015; A61K 9/0021; A61K 9/703; A61K 47/36; A61K 8/0208; A61K 8/735; B29L 2031/756; B29C 41/003; B29C 41/26; B29C 41/30; A61M 2037/0053; A61M 2037/0023; B29K 2089/00; B29C 41/32

(57) ABSTRACT

The present invention provides a microneedle patch of which continuous production is possible by a rotary method and in which a front end thereof is composed of hyaluronic acid, and a manufacturing method and a manufacturing device suitable for continuous production of the same.

The microneedle patch according to the present invention is one of which a myriad of needle-shaped parts composed of hyaluronic acid are pressed to be attached to a surface of an adhesive layer 4 laminated on a substrate 2, one of which part of the hyaluronic acid layer 8 which is coated on the surface of the adhesive layer 4 is formed into a needle-shaped part 6 so as to be pressed to be attached thereto, or one of which the hyaluronic acid layer 8 is laminated on the substrate 2 composed of a thin film and then pressed so as to be arranged into a myriad of needle-shaped parts 6. The microneedle patch is continuously manufactured by a method in which the adhesive layer 4 is coated on a substrate 2 by primary gravure coating followed by drying, and then an aqueous hyaluronic acid solution is coated by secondary gravure coating and pressed so as to form a needle-shaped part 6, or in which the hyaluronic acid layer 8 is coated on the substrate 2 and pressed so that part of the hyaluronic acid layer 8 is formed into a needle-shaped part 6. A manufacturing device which includes a first coating part 20 that coats the substrate 2 with an adhesive layer 4 or a hyaluronic acid layer 8, and a second coating part 30 including needle-forming rollers 33 and pressure rollers 35 that press the first drying part 26 and the substrate 2 so as to form a needle-shaped part 6, by a rotary method, is also included.

### TECHNICAL FIELD

The present invention relates to an apparatus and method for manufacturing a microneedle patch in which numerous microneedles for directly injecting drugs or cosmetic components by penetrating the skin are arranged on a thin substrate, and a microneedle patch manufactured by the same.

### BACKGROUND ART

Microneedle patches are attracting attention as a means of administering drugs or cosmetic components through the skin without pain, or as a tool for medical procedures such as blood sampling.

Microneedle patches have a structure in which a myriad of microneedles are protruded from one surface of a substrate, and for example, as disclosed in Patent Document 1 below, these microneedles are arranged in a range of 1 μm to 1 cm on a substrate which has a thickness of 1 μm to 1 cm and a width of 1 mm to 10 cm.

For materials forming these microneedles, as disclosed in Patent Document 2 below, biocompatible resins such as maltose, polylactic acid, dextran (i.e., a type of polysaccharide), etc., which are harmless to the human body, are employed.

Additionally, as a manufacturing method capable of implementing microneedles, methods such as electrochemical etching of silicon, plasma etching, electroplating, microlithography, etc., are known.

However, the above manufacturing method has problems in that it is not suitable for mass production, and thus efforts have been made to apply an injection molding method, an imprinting method, a casting method, etc. However, there are considerable limitations for performing continuous molding because the microneedles have their own standards requiring a high aspect ratio, a sharp tip, etc.

The following Patent Documents 3 and 4 disclose manufacturing methods with respect to the array of microneedles which are prepared in structures suitable for blood collection due to a long hole formed in the center of the tip.

The disclosed methods show processes in which a myriad of thin and lengthy grooves formed on the surface of a resist material at predetermined intervals in the horizontal and vertical directions by perforating toward the thickness direction are installed, and are then exposed to x-rays in the horizontal and vertical directions through an x-ray mask in the thickness direction and developed, while the surface in the shape of a microneedle array obtained therefrom is electroplated with nickel, etc. and the resist material is removed therefrom and thereby a microneedle forming mold in which microneedle-shaped concave grooves are arranged is obtained, and the microneedles are subjected to replication molding by the forming mold.

Unlike the methods described above, the following Patent Documents 5 and 6 disclose manufacturing methods of microneedles in which a mother mold where a myriad of microneedles are arranged on a substrate is prepared, and microneedles are manufactured using the forming mold on which the mother mold is replicated.

However, all of these manufacturing methods include coating a liquid raw material on a flat forming mold, drying, and skimming, and microneedles are produced by a stamper method.

Additionally, the following Patent Documents 7 and 8 disclose embodiments in which, at the time of performing the stamper method, when the microneedle-forming material is filled inside the lengthy groove of the forming mold, the microneedle-forming material is installed to perforate through the lengthy groove so that the air present inside of the forming mold can be released.

In another embodiment, the following Patent Document 9 discloses a manufacturing method in which, in a state where the molten resin is eluted at an end of a nozzle in an approximate semi-circular shape, if a substrate approaches thereto and is pressed to be attached and then pulled away, microneedles are formed while the molten resin is drawn.

Microneedles for cosmetic purposes are formed into a mixture of hyaluronic acid and a polymer material, as disclosed in the following Patent Documents 10 and 11. In this case, the physical properties of the microneedles vary greatly depending on the content of the polymer material, and thus the mixing ratio is limited to an appropriate range.

The cosmetic effects of the microneedles are mainly obtained from the hyaluronic acid.

However, since hyaluronic acid is expensive, the manufacturing cost increases as the amount of hyaluronic acid increases.

For the above reason, the following Patent Document 12 discloses a microneedle array that can minimize the manufacturing cost by forming the microneedles with an inexpensive polymer material and performing thin coating only on the tips of the needles.

However, the microneedle array using the above method not only causes deformation of a product such that as tip of a needle being twisted or dulled while the surface of the polymer material is being dissolved in water of an aqueous solution, but also the fraction of the coated hyaluronic acid is not sufficient to be administered into the skin to exhibit a cosmetic effect, and thus the product quality cannot be guaranteed.

The following Patent Document 13 discloses a microneedle array in which only the tips of microneedles are formed first with hyaluronic acid, maining part is formed with a polymer material, which is inexpensive.

### PRIOR ART DOCUMENTS

### Patent Documents

(Patent Document 1) U.S. Pat. No. 6,503,231
(Patent Document 2) Japanese Patent Laid-Open Publication No. 2005-21677
(Patent Document 3) Japanese Patent Laid-Open Publication No. 2005-246595
(Patent Document 4) Japanese Patent Laid-Open Publication No. 2009-61212
(Patent Document 5) Japanese Patent Laid-Open Publication No. 2009-273772
(Patent Document 6) Japanese Patent Laid-Open Publication No. 2010-94414
(Patent Document 7) Japanese Patent Laid-Open Publication No. 2011-78617
(Patent Document 8) Japanese Patent Laid-Open Publication No. 2011-78618
(Patent Document 9) Japanese Patent Laid-Open Publication No. 2011-5245
(Patent Document 10) Japanese Patent Laid-Open Publication No. 2010-29634
(Patent Document 11) Japanese Patent Laid-Open Publication No. 2010-82401
(Patent Document 12) International Patent Laid-Open Publication No. 2010-87300
(Patent Document 13) Japanese Patent Laid-Open Publication No. 2011-12050

### DISCLOSURE

### Technical Problem

An object of the present invention is to provide a microneedle patch manufacturing device which composed of components continuously form a microneedle patch in a long sheet to be produced in large quantities, in which needles composed of hyaluronic acid are infinitively arranged with integrated by an adhesive layer on a substrate.

Another object of the present invention is to provide a microneedle patch manufacturing device which can form a microneedle patch in a long continuous sheet to be produced in large quantities, in which needles composed of hyaluronic acid are infinitively arranged on a substrate.

Yet another object of the present invention is to provide a microneedle patch manufacturing method which composed of components continuously form a microneedle patch in a long continuous sheet to be produced in large quantities, in which needles composed of hyaluronic acid are infinitively arranged with integrated by an adhesive layer on a substrate.

Yet another object of the present invention is to provide a manufacturing method which can continuously form a microneedle patch, in which needles composed of hyaluronic acid are formed on a substrate.

Yet another an object of the present invention is to provide a microneedle patch by the manufacturing method, in which needles composed of hyaluronic acid are infinitively continuous arranged with integrated by an adhesive layer on a substrate.

Yet another object of the present invention is to provide a microneedle patch by the manufacturing method, in which needles composed of hyaluronic acid are formed on a substrate.

### Technical Solution

In order to implement the objects of the present invention, a manufacturing device of the microneedle patch according to the present invention consists of a first coating part, which is provided with a gravure roller that coats an adhesive layer on the lower surface of a substrate, and a first container in which an aqueous polymer material solution composed of the adhesive layer is contained, a first drying part in which the adhesive layer coated on the lower surface of the substrate is dried, and a second coating part, which is provided with a needle-forming roller which dispenses hyaluronic acid into a myriad of conical grooves formed on the external circumferential surface thereof and transferring while drying, a second container in which an aqueous hyaluronic acid solution is contained, a plurality of pressure rollers which are arranged to allow the adhesive layer of the substrate to come into close contact with the external circumferential surface of the needle-forming roller, a delivery roller that passes through the conical grooves and recovers the substrate to which the needle-shaped part is integrally attached, and a guide roller.

Additionally, a manufacturing device of the microneedle patch according to the present invention consists of a first coating part, which is provided with a gravure roller that coats a hyaluronic acid layer on the lower surface of a substrate composed of a thin film, and a first container in which an aqueous hyaluronic acid composed of the hyaluronic acid layer is contained, a first drying part in which the hyaluronic acid layer coated on the lower surface of the substrate is semi-dried, and a second coating part, which is provided with a needle-forming roller on which a myriad of conical grooves that allow part of the hyaluronic acid layer to be extruded to a needle-shaped part are formed on the external circumferential surface thereof; a plurality of pressure rollers which are arranged to come in close contact with the external circumferential surface of the needle-forming roller and thereby allowing the substrate to pass while pressurizing the substrate, a delivery roller that recovers a substrate on which the needle-shaped part is integrally formed, a guide roller, and a tape roll which supplies an adhesive tape to the opposite surface of the substrate by interposing the delivery roller.

In the manufacturing device of the microneedle patch according to the present invention consisting of the constitutions described above, a second drying path may be further disposed after the guide roller so as to fully dry the needle-shaped part being formed by passing through the needle-forming roller.

In the manufacturing device of the microneedle patch described above, it is preferred that the delivery roller not only be disposed at predetermined intervals by being shifted to an arrangement angle with respect to the needle-forming roller or the external circumferential surface of the needle-forming roller, but to also be disposed at a lower position than the position where the needle-shaped part escapes from the conical grooves at the height of the horizon past the center of the needle-forming roller.

A method for manufacturing a microneedle patch using an apparatus for manufacturing a microneedle patch described above is obtained by performing gravure coating of an adhesive layer on a substrate followed by drying, filling an aqueous hyaluronic acid solution into the conical grooves of a needle-forming roller followed by semi-drying thereby forming a needle-shaped part, and then pressing the adhesive layer of the substrate to the needle-forming roller thereby transferred so that a part of the adhesive layer is pressed into the conical grooves thereby integrally attached to the needle-shaped part thereby continuously producing the substrate in a sheet shape where the needle-shaped part is infinitely formed.

In addition, a method for manufacturing a microneedle patch using an apparatus for manufacturing a microneedle patch described above is obtained by performing gravure coating of a hyaluronic acid layer on a substrate which is comprised of a thin film followed by semi-drying, pressing the substrate to an external circumference of the needle-forming roller thereby transferred so that part of the semi-dried hyaluronic acid layer is extruded to a conical groove of the needle-forming roller to be formed into a needle-shaped part; and then attaching an reinforcing tape to an opposite side of the substrate thereby continuously producing the substrate in a sheet shape where the needle-shaped part is infinitely formed.

In addition, in a microneedle patch obtained by the method for manufacturing a microneedle patch, part of an adhesive layer which is coated and dried on one side of the upper surface of a substrate including a film or non-woven fabric is extruded by a pressure thereby integrally attached with needle-shaped parts.

Additionally, the microneedle patch according to the present invention has a constitution which includes: an adhesive layer which is coated on one side of the upper surface of a substrate including a film or non-woven fabric on a flat plate and integrally attached thereto; a hyaluronic acid layer which is laminated on the upper surface of the adhesive layer; and a needle-shaped part on which a myriad of parts of the hyaluronic acid layer are extruded and arranged thereon.

Additionally, the microneedle patch according to the present invention has a constitution which is provided with: a hyaluronic acid layer which is coated on one side of the surface of a substrate including a thin film on a flat plate and semi-dried; a myriad of needle-shaped parts which are extruded to the upper surface of the hyaluronic acid layer and arranged thereon; and an adhesive tape which is attached to the opposite surface of the substrate to reinforce the thin film to prevent distortion.

In the microneedle patch described above, the adhesive layer is formed of a polymer material and the needle-shaped part is formed of hyaluronic acid.

For the polymer material, one kind or a mixture of two or more kinds of a water-soluble or water-expandable resin selected from the group consisting of polysaccharides such as glycogen, dextrin, dextran, dextran sulfate, sodium chondroitin sulfate, hydroxypropyl cellulose, chitosan, alginic acid, agarose, chitin-chitosan, pullulan, amylopectin, glycerin, starch, etc.; proteins such as collagen, gelatin, albumin, etc.; and synthetic polymers such as polyvinyl alcohol (PVA), carboxyvinyl polymers, sodium polyacrylate, polyvinylpyrrolidone, polyethylene glycol, etc., and among them, collagen, gelatin, polyvinylpyrrolidone, and polyethylene glycol may be adopted.

For hyaluronic acid, culture-derived hyaluronic acid that can be obtained from lactobacteria, Streptococcus, etc. may be employed.

Additionally, one kind or a mixture of two or more kinds selected from an AHA such as glycolic acid, lactic acid, etc., tocopherol, and adenosine may be added to the hyaluronic acid.

### Advantageous Effects

The manufacturing device of the microneedle patch according to the present invention may have a good work effectiveness and be produced in large quantities thereby powerfully reducing the manufacturing cost of the microneedle patch, because a microneedle patch may be continuously formed in which needles composed of hyaluronic acid are infinitively arranged with integrated on a substrate of a long extended sheet-shape.

The manufacturing method of the microneedle patch according to the present invention may have advantages in that it is convenient to manage processes and it is automatically produced thereby powerfully reducing labor force, because processes may be performed by the lump which an adhesive layer or a hyaluronic acid layer is coated on a substrate, and a needle-shaped part is integrally adhesive or formed.

The microneedle patch according to the present invention may have advantages in that it may be obtained by cutting out a substrate of a long extended sheet shape produced in large quantities thereby powerfully reducing a manufacturing cost, and it powerfully reduces the manufacturing cost and it does not undergo exterior deformation because a hyaluronic acid layer is directly laminated and pressed on a substrate composed of a particularly thin film so that a reinforcing tape which is attached on opposite surface where needle-shaped parts are infinitely extruded and arranged, may protect from deformation or curling of the substrate thereby reducing a thickness of the expensive hyaluronic acid layer at a minimum level.

### DESCRIPTION OF THE DRAWINGS

FIG. 1 is a schematic diagram showing a manufacturing device of a microneedle according to an embodiment of the present invention.

FIG. 2 is an enlarged cross-sectional view of a major part of the manufacturing device shown in FIG. 1.

FIG. 3 is a cross-sectional side view showing an embodiment of a microneedle patch manufactured by the device of FIG. 1.

FIG. 4 is a cross-sectional side view showing another embodiment of a microneedle patch manufactured by the device of FIG. 1.

FIG. 5 is a schematic diagram showing a manufacturing device of a microneedle according to another embodiment of the present invention.

FIG. 6 is a cross-sectional side view showing another embodiment of a microneedle patch manufactured by the device of FIG. 5.

FIG. 7 is an enlarged image showing the real of the microneedle patch shown in FIG. 6.

FIG. 8 is a partially-enlarged diagram showing another embodiment of a needle-forming roller, in the manufacturing device according to the present invention.

### MODE FOR INVENTION

The present invention will now be described in detail as a preferred embodiment according to the accompanying drawings.

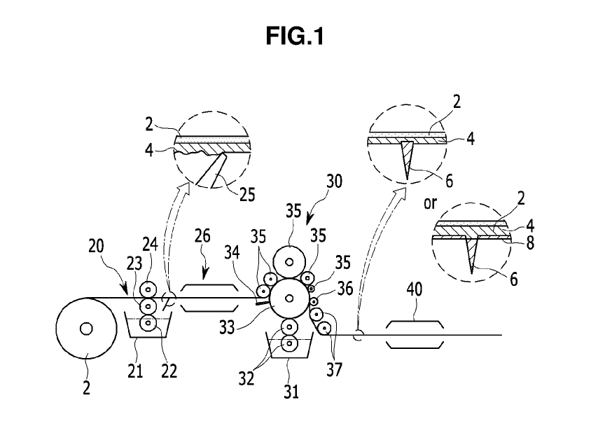

The manufacturing device of a microneedle patch according to the present invention, as shown in FIG. 1, has a constitution consisting of a first coating part 20, a first drying part 26, a second coating part 30, and a second drying part 40, which are sequentially arranged.

The first drying part 26 may be dried by blowing heated air or dehumidified air therein or by an evaporation effect, however, it is preferred for the second drying part 40, which is for drying the final product, to be dried by blowing a hot air therein.

The first coating part 20 has a constitution where a first container 21 in which a polymer material, hot-melt, or medical adhesive agent is contained, a first transfer roller 22 which is installed on the first container 21 so that part of the external circumferential surface can be dipped into the first container 21, a gravure roller 23 which is disposed to be able to rotate by coming into contact with the first transfer roller 22, and a backup roller 24 which rotates by opposing and coming into contact with the gravure roller 23, are installed.

The polymer material contained in the first container 21 is supplied to the surface of the gravure roller 23 by interposing the first transfer roller 22, the substrate 2 passes between the gravure roller 23 and the backup roller 24, and thereby the polymer material smeared onto the surface is coated to the lower surface of the substrate 2 and its thickness is controlled through a first doctor blade 25, thereby forming the adhesive layer 4.

The substrate 2 on which the adhesive layer 4 is coated on the lower surface thereof is dried while passing through the first drying part 26 and then passes through the second coating part 30.

The second coating part 30 is provided with a second container 31 in which an aqueous hyaluronic acid solution is contained, similar to the first coating part 20, a second transfer roller 32 which is disposed in the second container 31, and a needle-forming roller 33 which is installed to rotate by coming into contact with the second transfer roller 32.

The needle-forming roller 33 is a hot roller that always maintains a constant temperature by circulation such as hot as, steam, etc., through a built-in heater or an internal hollow passage.

Additionally, a second doctor blade 34 is disposed on the external circumference of the needle-forming roller 33, and thus hyaluronic acid being coated is wiped off to prevent hyaluronic acid from remaining on the surface, or controls so that the hyaluronic acid layer 8 is coated to a constant thickness, and simultaneously, on a pathway which does not hinder the movement of the substrate 2, a plurality of large and small pressure rollers 35 are arranged to rotate by coming into close contact with the external circumference of the needle-forming roller 33.

Additionally, continuously, a delivery roller 36 and a guide roller 37 are sequentially arranged toward the front of the second drying part 24.

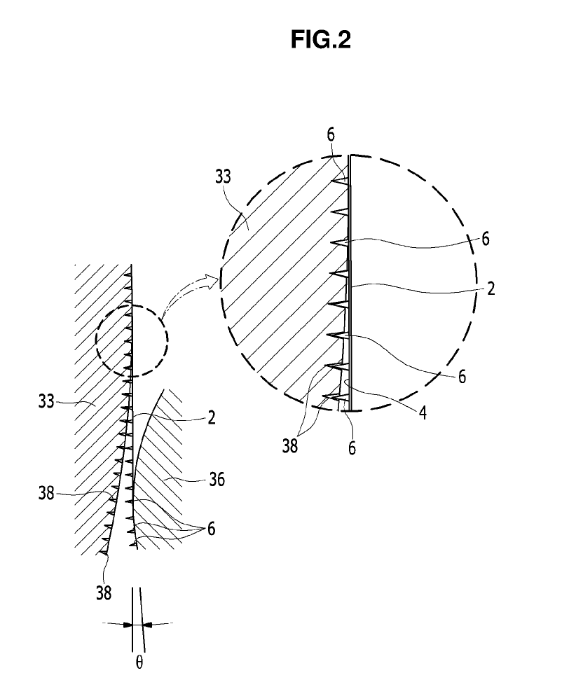

The needle-forming roller 33, as shown in FIG. 2, is one in which a myriad of conical grooves are formed on the external circumferential surface thereof.

When the substrate 2 which is transported through the first drying part 26 passes through the second coating part 30, the adhesive layer 4 coated on the lower surface thereof comes into contact with the surface of the needle-forming roller 33, and subsequently passes while being pressed through the large and small pressure rollers 35.

Meanwhile, when the second doctor blade 34 is controlled so that hyaluronic acid does not remain on the external circumferential surface of the needle-forming roller 33, the coated hyaluronic acid is filled into conical grooves 38 and is transported while being dried, whereas when the second doctor blade 34 is controlled to coated the hyaluronic acid layer 8 with a certain thickness, the adhesive layer of the substrate 2 that comes into contact with the external circumferential surface of the needle-forming roller 33 is coated with the hyaluronic acid layer 8.

Further, when the substrate 2 is pressed by a plurality of pressure rollers 35 on the external circumferential surface of the needle-forming roller 33, if hyaluronic acid which is filled into the conical grooves 38 and being dried is pressed and attached to the surface of the adhesive layer 4 and thereby forms the needle-shaped part 6, or if coated with the hyaluronic acid layer 8, the part corresponding to the boundary of the conical groove 38, exclusive of the hyaluronic acid layer 8, is pressed and simultaneously the adhesive layer 4 and the hyaluronic acid inside of the conical groove 38 are integrally attached to each other thereby forming the needle-shaped part 6.

The substrate 2 in which the needle-shaped part 6 is formed as such is dried while going around the external circumferential surface of the needle-forming roller 33, finally is transferred to the delivery roller 36 and thereby departs from the needle-forming roller 33 and is recovered along the guide roller 37.

Preferably, the needle-shaped part 6 in the substrate 2 being recovered is in a state of not being completely dried, and thus it is desirable for the needle-shaped part to immediately pass through the second drying part 40 to be completely dried.

The delivery roller 36 must be disposed such that the needle-shaped part 6 does not undergo deformation by interference when the substrate 2 is transferred in the needle-forming roller 33.

That is, the interference of the needle-shaped part 6 occurs because the angle between the conical groove 38 and the substrate 2 continuously changes by the rotation of the needle-forming roller 33 while the departure angle of the substrate 2 is constant in the process of departing from the conical groove 38 of the needle-forming roller.

In the present invention, to minimize the deformation of the needle-shaped part 6 described above, the delivery roller 36 is installed with a minute interval from the external circumferential surface of the needle-forming roller 33 and is arranged to be shifted by the difference of the disposed angle (θ).

In addition, the center of the delivery roller 36 is disposed lower than the position where the needle-shaped part 6 begins a rotational movement at the conical groove 38 located on the horizon that passes through the center of the needle-forming roller, and thereby preferably prevents interference when the needle-shaped part 6 is departed from the conical groove 38.

However, the space between the needle-forming roller 33 and the delivery roller 36 may be adjusted according to the height of the microneedle desired to be obtained.

Meanwhile, in the needle-forming roller 33, the conical groove 38 may actually adopt one in which the external circumferential surface of a Teflon tube is molded by laser or endmill processing, and a Teflon tube in which the conical groove 38 is formed can be obtained if a circular roller in which artificial conical diamond particles selected with a uniform size are attached to the surface thereof is attached face-to-face to the external circumferential surface of a Teflon tube and is allowed to rotate while applying a pressure thereto.

Next, the manufacturing method of a microneedle patch will be explained.

A desired microneedle patch may be obtained by coating one kind selected from a polymer material, a hot-melt, and a medical adhesive agent on the substrate 2 by primary gravure coating, drying, laminating the adhesive layer 4 thereon, and drying and pressurizing while coating hyaluronic acid containing two or more kinds of materials selected from a low molecular weight materials, polysaccharides, monosaccharides, and functional materials, on the adhesive layer 4 by secondary gravure coating, thereby allowing the hyaluronic acid is formed into the needle-shaped part 6 and integrally attached to the surface of the adhesive layer 4; or a desired microneedle patch may be obtained by drying and pressurizing while coating the hyaluronic acid layer 8 on the surface of the adhesive layer 4 by secondary gravure coating, thereby allowing part of the hyaluronic acid layer 8 to be extruded and integrally attached to the adhesive layer 4.

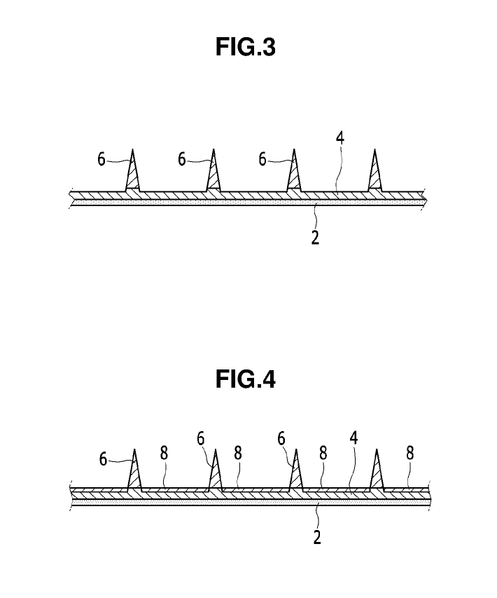

The microneedle patch obtained by the above method, in case that an aqueous hyaluronic acid solution is filled only into the conical groove 38, as shown in FIG. 3, consists of an adhesive layer 4 which is coated on one side of the upper surface of a substrate 2 composed of a film, non-woven fabric, etc., and integrally attached thereon on a flat plate, and also, as shown in FIG. 4, a projected part of the adhesive layer 4 which are passed through the hyaluronic acid layer 8 is integrally attached with the needle-shaped part 6.

In an exemplary embodiment, the adhesive layer 4 is composed of a polymer material, and the needle-shaped part 6 is composed of hyaluronic acid or a mixture of hyaluronic acid and a cosmetic component, or a mixture of hyaluronic acid and a medicinal component.

The polymer material that can be adopted in the microneedle patch of the present invention may be adopted by selecting from polysaccharides such as glycogen, dextrin, dextran, dextran sulfate, sodium chondroitin sulfate, hydroxypropyl cellulose, chitosan, alginic acid, agarose, chitin-chitosan, pullulan, amylopectin, glycerin, starch, etc.; proteins such as collagen, gelatin, albumin, etc.; or synthetic polymers such as polyvinyl alcohol (PVA), carboxyvinyl polymer, sodium polyacrylate, polyvinylpyrrolidone, polyethylene glycol, etc., and may be implemented using, among them, one kind or a mixture of two or more kinds selected from collagen, gelatin, polyvinylpyrrolidone, and polyethylene glycol.

Hyaluronic acid adopts culture-derived hyaluronic acid, and if necessary, an appropriate amount of a cosmetic component such as AHA (e.g., glycolic acid, lactic acid, etc.) which is used for dieting, whitening, wrinkle improvement, acne improvement, and keratin improvement; tocopherol, adenosine, etc., or a medicinal component such as insulin, nicotine, a vaccine, etc., may be added to the hyaluronic acid.

In the microneedle patch of the present invention described above, only the needle tips that penetrate through the skin are composed of expensive hyaluronic acid and the remaining part thereof is composed of an inexpensive polymer material, and thus the microneedle patch of the present invention can reduce the manufacturing cost to a minimal level and also supply a sufficient amount of hyaluronic acid that can penetrate through the skin and be absorbed therein.

When the adhesive layer 4 on a flat plate and the conical needle-shaped part 6 are pressed and integrally attached on the substrate 2, the adhesive layer 4 is attached while pressing toward the needle-shaped part 6 and thereby the needle-shaped part 6 is formed into a dense structure having no air bubbles inside.

If the adhesive layer 4, after being coated on the substrate 2 and sufficiently dried, is allowed to come into close contact with the needle-shaped part 6, the pressure being imparted to the needle-shaped part 6 is not reduced and thus a good adhesion state can be obtained.

In the present invention, the adhesive layer 4 can adopt a polymer material, hot-melt, or medical adhesive agent, and the adhesive layer 4 is reinforced by the substrate 2 which is composed of a non-woven fabric, film, etc. and thus has a significant level of tensile strength.

Additionally, as part of the region in the adhesive layer 4 that comes into contact with the needle-shaped part 6 is extruded due to the action of an external pressure being applied to the region which does not come into contact with the needle-shaped part 6 on the upper surface of the adhesive layer 4, the shape that supports the needle-shaped part 6 is integrally attached while undergoing deformation and is thereby formed to have an external appearance having a characteristic represented in FIG. 3 or FIG. 4.

Meanwhile, the microneedle patch shown in FIG. 4 is consisted where the upper surface of the adhesive layer 4 is covered by a hyaluronic acid layer 8 other parts expert from the needle-shaped part 6.

In this case, the hyaluronic acid layer 8 that remains on the upper surface of the adhesive layer 4 in the periphery of the needle-shaped part 6 comes into contact with the skin when it is used and is thus dissolved and absorbed by the action of body temperature, and thereby an improvement of a cosmetic effect by the same is expected.

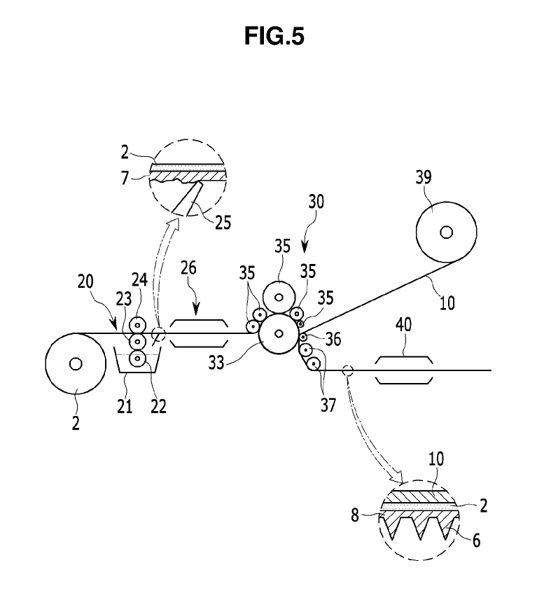

The microneedle patch manufacturing device according to another embodiment of the present invention shown in FIG. 5 has basically the same constitution as that shown in FIG. 1.

More specifically, the first coating part 20 includes a constitution which is provided with a first container 21, a first transfer roller 22 which is installed such that part of the external circumferential surface thereof is dipped into the first container 21, a gravure roller 23 which is disposed to come into contact with the first transfer roller 22 and rotate, and a backup roller 24 which is installed to come into contact with the gravure roller 23 and rotate, but an aqueous hyaluronic acid solution is contained in the first container 21.

The aqueous hyaluronic acid solution contained in the first container 21 is coated on the surface of the gravure roller 23 by interposing the first transfer roller 22, the aqueous hyaluronic acid solution smeared on the surface is coated to the lower surface of the substrate 2 when passing between the gravure roller 23 and the backup roller 24, and the thickness is adjusted through the first doctor blade 25 thereby forming the hyaluronic acid layer 8.

The substrate 2 coated with the hyaluronic acid layer 8 on the lower surface is semi-dried while passing through the first drying part 26.

Also, the hyaluronic acid layer 8 being coated to the substrate 2 can be extruded and formed into the needle-shaped part 6 when it is semi-dried.

That is, in this exemplary embodiment, the second coating part 30 has a constitution provided with a needle-forming roller 33, a pressure roller 35 at its periphery, a delivery roller 36, and a guide roller 37 and has not the second container 31 and the second transfer roller 32.

Accordingly, when the substrate 2 that is semi-dried by going through the first drying part 26 passes through the second coating part 30, the hyaluronic acid layer coated on the lower surface thereof comes into contact with the surface of the needle-forming roller 33 and moves while being pressed by a plurality of pressure rollers 35.

Additionally, as a result of the hyaluronic acid layer being pressed by the plurality of pressure rollers 35 on the external circumferential surface of the needle-forming roller 33, part of the hyaluronic acid layer 8 is extruded and forms a needle-shaped part 6 on the conical groove 38 of the needle-forming roller 33.

Then, the substrate 2 is transferred into the delivery roller 36 while forming the needle-shaped part 6 and changes its pathway along the guide roller 37 and departs from the needle-forming roller 33 to be recovered.

Although a myriad of needle-shaped parts 6 are integrally formed on the surface of the hyaluronic acid layer 8 of the substrate 2 being recovered, the hyaluronic acid layer 8 is not completely dried and thus it is preferred that the hyaluronic acid layer 8 be finally dried by passing through the second drying part 40.

Additionally, in the manufacturing device described above, the substrate 2 and the hyaluronic acid layer 8 of thereon are set as a minimum thickness, in this case, the patch thus causes a deformation of being curled due to the contractive action of the dried.

In this exemplary embodiment, to prevent the substrate 2 from being curled, an adhesive tape 10 rolled on an adhesive tape roll 39 is supplied through the delivery roller 36 and attached to the opposite surface of the substrate 2 to reinforce the morphological integrity, thereby avoiding being curled.

The manufacturing device of the microneedle patch described above obtains a desired microneedle patch by that the hyaluronic acid layer 8 is applied to the substrate 2 by primary gravure coating and semi-dried, is passed between the needle-forming roller 33 and the pressure roller 35, and part of the hyaluronic acid layer 8 is extruded and formed into the needle-shaped part 6.

The microneedle patch obtained by the above embodiment has the morphological integrity because part of the hyaluronic acid layer 8 is extruded and formed into the needle-shaped part 6 and the reinforced tape 10 is attached to the opposite surface of the substrate 2

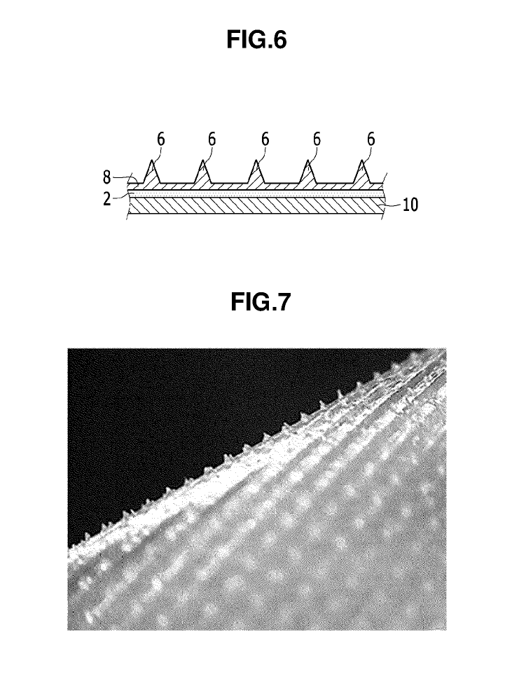

The real microneedle patch shown in FIG. 6 is c. as shown in FIG. 7 has the hyaluronic acid layer 8 consisted of the flat surface and the needle-shaped parts 6 in form continuously extruded at surroundings thereof, and the inner small photograph of in FIG. 7 enlarges one of the needle-shaped parts 6.

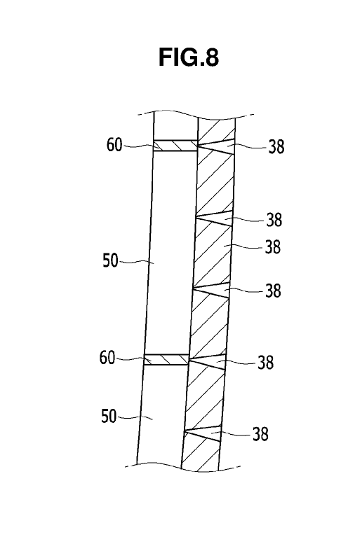

FIG. 8 shows another example of the conical groove 38 of the needle-shaped roller 33 described above.

The conical groove 38 may be a perforated hole. In this case, the perforated hole may be a passage for internal air to be released therethrough when an aqueous hyaluronic acid solution is filled into the conical groove 38 thereby the needle-shaped part 6 is formed to have high density and a good external appearance.

Additionally, in the type which the conical groove 38 may be a perforated hole, a reinforcing tube body 60 having an exhaust hole 50 may be provided on the inside thereof, if necessary.

As described above, the present invention has a different concept with the prior method which the aqueous hyaluronic acid solution etc. is filled into a mold of stamping type, stamped thereby forming the microneedles and substrate part, separated and recovered from the mold while drying because it continuously produce microneedles of a long extended sheet shape thereby cutting out them at a predetermined size and providing a microneedle patch as goods.

That is, the present invention may continuously manufacture the desired microneedle patch by that the substrate is passed between the needle-forming roller and the pressure roller in the manufacturing apparatus having a plurality of pressed rollers which is disposed to be pressed at the periphery thereof and come into contact to be able to rotate with the needle-forming roller which conical grooves are infinitely formed on an external circumference thereof

Also, the method for manufacturing a microneedle patch may continuous produce a microneedle patch where the part of the adhesive layer is pressed and extruded into the conical grooves thereby integrally attached to the needle-shaped part, in case that the adhesive layer is coated on the substrate and the hyaluronic acid is filled into the conical grooves of a needle-forming roller thereby forming the needle-shaped part, or may continuous produce a microneedle patch where the part of the adhesive layer presses the hyaluronic acid layer into the conical grooves and is passed therethrough thereby forming the needle-shaped part and simultaneously integrally attached to the needle-shaped part, in case that the adhesive layer is coated on the substrate and the hyaluronic acid is coated on an external circumference surface of the needle-forming roller.

In addition, the method for manufacturing a microneedle patch may continuous produce a microneedle patch where in case that the hyaluronic acid layer is coated on the substrate, part of the hyaluronic acid layer are extruded into the conical grooves of the needle-forming roller by pressure thereby forming the needle-shaped part, the reinforced tape is attached to an opposite surface of the substrate so to have a morphological integrity.

### DESCRIPTION OF SYMBOLS

| | |
|---|---|
| 2: substrate | 4: adhesive layer |
| 6: needle-forming layer | 8: hyaluronic acid layer |
| 10: adhesive tape | 20: first coating part |
| 21: first container | 22: first transfer roller |
| 23: gravure roller | 24: first backup roller |
| 25: first doctor blade | 26: first drying part |
| 30: second coating part | 31: second container |
| 32: second transfer roller | 33: needle forming roller |
| 34: second doctor blade | 35: pressure roller |
| 36: delivery roller | 37: guide roller |
| 38: conical groove | 39: adhesive tape roll |
| 40: second drying part | 50: exhaust hole |
| 60: reinforcing tube body | |

1. An apparatus for manufacturing a microneedle patch, comprising:
a first coating part, which is provided with a first container in which one selected from an aqueous polymer material solution and a hot-melt medical adhesive agent is contained; a first transfer roller which is installed so that part of the external circumferential surface thereof is dipped into the first container; and a gravure roller which is disposed to come into contact with the first transfer roller to be able to rotate;
a first drying part which is connected to the first coating part; and
a second coating part, which is provided with a second container in which an aqueous hyaluronic acid solution is contained; a second transfer roller disposed therein; a needle-forming roller where a myriad of conical grooves are formed on the external circumferential surface thereof and which always maintain a heated state; and a second doctor blade, a plurality of pressure rollers, a delivery roller, and a guide roller, which are sequentially arranged on the external circumference of the needle-forming roller.

2. An apparatus for manufacturing a microneedle patch, comprising:
a first coating part, which is provided with a first container in which an aqueous hyaluronic acid solution is contained; a first transfer roller which is installed so that part of the external circumferential surface thereof is dipped into the first container; and a gravure roller which is disposed to come into contact with the first transfer roller to be able to rotate;
a first drying part which is connected to the first coating part; and
a second coating part, which consists of a needle-forming roller, in which a myriad of conical grooves are formed on the external circumferential surface thereof and which is always maintained in a heated state, and a plurality of pressure rollers, a delivery roller, and a guide roller, which are sequentially arranged on the external circumference of the needle-forming roller.

3. The apparatus of claim 1, wherein
a second drying part is further disposed while being connected to the second coating part.

4. A method for manufacturing a microneedle patch using an apparatus for manufacturing a microneedle patch of claim 1, which is obtained by: performing gravure coating of an adhesive layer on a substrate followed by drying, filling an aqueous hyaluronic acid solution into the conical grooves of a needle-forming roller followed by semi-drying thereby forming a needle-shaped part, and then pressing the adhesive layer of the substrate to the needle-forming roller thereby transferred so that a part of the adhesive layer is pressed into the conical grooves thereby integrally attached to the needle-shaped part thereby continuously producing the substrate in a sheet shape where the needle-shaped part is infinitely formed.

5. A method for manufacturing a microneedle patch using an apparatus for manufacturing a microneedle patch of claim 1, which is obtained by: performing gravure coating of a hyaluronic acid layer on a substrate which is comprised of a thin film followed by semi-drying; pressing the substrate to an external circumference of the needle-forming roller thereby transferred so that part of the semi-dried hyaluronic acid layer is extruded to a conical groove of the needle-forming roller to be formed into a needle-shaped part; and then attaching an reinforcing tape to an opposite side of the substrate thereby continuously producing the substrate in a sheet shape where the needle-shaped part is infinitely formed.

6. The Method of claim 4, wherein the adhesive layer of the substrate is pressed and extruded to the needle-forming roller so that the part of the adhesive layer is pressed into the conical grooves passing though the hyaluronic acid layer thereby forming the needle-shaped part and simultaneously integrally attached to the adhesive layer thereby continuously producing the substrate in a sheet shape where the needle-shaped part is infinitely formed.

7. A microneedle patch manufactured by the method for manufacturing a microneedle patch of claim 5 wherein the hyaluronic acid layer is coated on one side of the upper surface of a substrate thereby forming the needle-shaped parts, which are extruded by pressure on the upper surface of the hyaluronic acid layer, and a reinforced tape is attached to the substrate so to have a morphological integrity and consist of in a sheet shape where the needle-shaped part is infinitely formed.

8. The apparatus for manufacturing a microneedle patch of claim 6, wherein
the adhesive layer is formed of one or more kinds of polymer materials selected from the group consisting of polysaccharides such as glycogen, dextrin, dextran, dextran sulfate, sodium chondroitin sulfate, hydroxypropylcellulose, chitosan, alginic acid, agarose, chitin-chitosan, pullulan, amylopectin, glycerin, starch, etc.; proteins such as collagen, gelatin, albumin, etc.; polyvinyl alcohol, carboxyvinyl polymer, sodium polyacrylate, polyvinylpyrrolidone, and polyethylene glycol; or a hot-melt or medical adhesive agent.

9. The apparatus of claim 2, wherein
a second drying part is further disposed while being connected to the second coating part.

10. A method for manufacturing a microneedle patch using an apparatus for manufacturing a microneedle patch of claim 2, which is obtained by: performing gravure coating of an adhesive layer on a substrate followed by drying, filling an aqueous hyaluronic acid solution into the conical grooves of a needle-forming roller followed by semi-drying thereby forming a needle-shaped part, and then pressing the adhesive layer of the substrate to the needle-forming roller thereby transferred so that a part of the adhesive layer is pressed into the conical grooves thereby integrally attached to the needle-shaped part thereby continuously producing the substrate in a sheet shape where the needle-shaped part is infinitely formed.

11. A method for manufacturing a microneedle patch using an apparatus for manufacturing a microneedle patch of claim 2, which is obtained by: performing gravure coating of a hyaluronic acid layer on a substrate which is comprised of a thin film followed by semi-drying; pressing the substrate to an external circumference of the needle-forming roller thereby transferred so that part of the semi-dried hyaluronic acid layer is extruded to a conical groove of the needle-forming roller to be formed into a needle-shaped part; and then attaching an reinforcing tape to an opposite side of the substrate thereby continuously producing the substrate in a sheet shape where the needle-shaped part is infinitely formed.

12. The Method of claim 10, wherein the adhesive layer of the substrate is pressed and extruded to the needle-forming roller so that the part of the adhesive layer is pressed into the conical grooves passing though the hyaluronic acid layer thereby forming the needle-shaped part and simultaneously integrally attached to the adhesive layer thereby continuously producing the substrate in a sheet shape where the needle-shaped part is infinitely formed.

13. A microneedle patch manufactured by the method for manufacturing a microneedle patch of claim 11, wherein the hyaluronic acid layer is coated on one side of the upper surface of a substrate thereby forming the needle-shaped parts, which are extruded by pressure on the upper surface of the hyaluronic acid layer, and a reinforced tape is attached to the substrate so to have a morphological integrity and consist of in a sheet shape where the needle-shaped part is infinitely formed.

14. The apparatus for manufacturing a microneedle patch of claim 12, wherein
the adhesive layer is formed of one or more kinds of polymer materials selected from the group consisting of polysaccharides such as glycogen, dextrin, dextran, dextran sulfate, sodium chondroitin sulfate, hydroxypropylcellulose, chitosan, alginic acid, agarose, chitin-chitosan, pullulan, amylopectin, glycerin, starch, etc.; proteins such as collagen, gelatin, albumin, etc.; polyvinyl alcohol, carboxyvinyl polymer, sodium polyacrylate, polyvinylpyrrolidone, and polyethylene glycol; or a hot-melt or medical adhesive agent.
